(19) United States
(12) Patent Application Publication
(10) Pub. No.: US 2025/0345210 A1
(43) Pub. Date: Nov. 13, 2025

(54) Wound Dressing

(71) Applicant: Pellis Innovations Limited, Edinburgh (GB)

(72) Inventor: Brian James McCormack, Cluny By Kirkcaldy (GB)

(21) Appl. No.: 19/276,177

(22) Filed: Jul. 22, 2025

Related U.S. Application Data
(63) Continuation-in-part of application No. 18/510,762, filed on Nov. 16, 2023.
Application No. 18/510,762 is a division of application No. 16/604,884, filed on Oct. 11, 2019, now abandoned.

(30) Foreign Application Priority Data
Apr. 11, 2017 (GB) 1705800.9
Jul. 4, 2017 (GB) 1710771.5

(51) Int. Cl. A61F 13/0246; A61F 13/00; A61F 13/02; A61L 15/22; A61L 15/58

(52) U.S. Cl. CPC A61F 13/0253; A61F 13/0256; A61F 13/0276; A61L 15/22; A61L 15/58; A61F 2013/00361

(57) ABSTRACT

The present invention is directed to a wound dressing, surgical tape or plaster, optionally comprising an absorbent pad, and an adhesive part in which the adhesive part is an adhesive dissolvable paper or polymer.

### RELATED APPLICATIONS

This application is a continuation-in-part patent application claiming priority in divisional patent application Ser. No. 18/510,762 filed on Nov. 16, 2023, which in turn claims priority to U.S. patent application Ser. No. 16/604,884 filed on Oct. 11, 2019, which is the national stage of international patent application no. PCT/GB2018/050957 filed on Apr. 10, 2018, which in turn claims priority from Great Britain Patent Application Nos. 1705800.9 filed on Apr. 11, 2017 and 1710771.5 filed on Jul. 4, 2017, the disclosures of which are incorporated herein by reference in their entirety.

### TECHNICAL FIELD

The present invention relates to a wound dressing, surgical tape, cannula dressing, bandage or plaster.

### BACKGROUND

Wound dressings, surgical tape, bandages and plasters are widely used to cover wounds, hold together wounds or cover simple cuts. Previously proposed wound dressings, surgical tape, bandages and plasters have used sticky fabric, adhesive plastic film and such to attach them to a patient's skin. In the case of a wound dressing or plaster the sticky fabric, adhesive plastic film holds an absorbent pad, often impregnated with anti-septic or other topical medicine, on the wound or skin of the patient. Such pads are used to absorb leaking fluid or protect against foreign bodies entering the wound and necessarily the adhesion is designed to hold the pad securely against the skin. It is normally necessary to replace such plasters or dressings regularly due to leakage of fluid from the wound with a fresh dressing or a different type of dressing. The replacement of such a wound dressing or plaster involves tugging off the old plaster or dressing thereby often causing further damage to the wound. In the case of surgical tape, for instance, it is often used in operations to hold people's eyes shut during an operation and removing the tape, particularly from elderly patients, causes distress.

A cannula is a hollow tube with a sharp, retractable inner core that can be inserted into a vein, an artery, or another body cavity. It is generally covered with a specific dressing to hold it in place and stop the patient from accidentally disturbing it.

When removing a traditional cannula dressing, there can be a pain and discomfort due to the skin being pulled whilst the cannula is still in place in the vein.

Additionally, surgical tape is often used when performing cosmetic improvements, such as, but without limitation, when compressing or positioning skin to smooth out fine lines and wrinkles or post-operative, after removing sections of skin or performing face lifts. It is often uncomfortable to remove conventional tape as the tearing procedure can be painful or even counter-productive, having regard to the nature of the cosmetic procedure.

It is an aim of the present invention to provide a wound dressing, surgical tape, cannula dressing, bandage or plaster which seeks to overcome these previously mentioned disadvantages.

Accordingly the present invention is directed to a wound dressing, surgical tape or plaster in which the adhesive part comprises adhesive soluble paper or polymer. The water-soluble part can be easily removed via wiping with a wet sponge/cloth or even running water leaving little or no residue.

### SUMMARY

In a preferred embodiment the wound dressing or plaster, the adhesive soluble paper or polymer part optionally covers an absorbent pad. This provides the advantage that the adhesive paper or polymer can attach to the absorbent pad as well as to the skin of the person being treated. The soluble paper or polymer can then be removed without disturbing the absorbent pad or dressing or skin. This can then be gently removed with appropriate care to limit damage to the wound.

Advantageously the wound dressing, surgical tape or plaster has a removable film/foil covering its adhesive side before use. This enables the film/foil to be removed before use of the wound dressing, surgical tape or plaster.

Preferably the dissolvable paper or polymer can be printed or written or on one side conveying information such as the procedure in process or instructions for the removal of the paper or polymer.

Advantageously the wound dressing is covered by a removable water resistance film. This provides the advantage that if the wound dressing is accidentally splashed with water while in use it does not start to dissolve.

In a preferred embodiment the film is secured to that intended topside of the wound dressing via adhesive that is relatively weak to enable easy removal. Advantageously the film as a corner or edge that bears no adhesive in order to enable easy peel off before removal of the dressing.

Preferably the absorbent pad of the wound dressing is made of water-soluble material. The water-soluble material may be similar to, or the same as, that of the rest of the wound dressing or of a different material. This provides the additional advantage that when removing the top of the wound dressing the absorbent pad can also be gently washed away thus dealing with any adhesion of the pad to sutures or the like.

Preferably, the wound dressing is a cannula dressing. This dressing is advantageous for a short-term treatment and when the patient is bed-ridden, especially for burn injuries. Preferably, the cannula dressing is made from two forms of soluble material.

Advantageously, the cannula dressing can be made to any size or shape.

In a preferred embodiment, the cannula dressing can be secured to the cannula site.

Preferably, the cannula dressing can be flushed or washed away by water/saline spray or wet sponge.

Preferably, the wound dressing is a tape made of a paper or polymer material, ideally of elongate form.

A further aspect of the present invention is directed to a bandage of elongate form in which sections of the bandage can be of a woven or non-woven fabric, comprising multiple sections, wherein the sections are held together via water-soluble fibre material. This provides the advantage that if a wound dressing is covered by a standard elongate bandage the bandage can be removed carefully particularly when soiled in sections.

In a preferred embodiment the sections are sewn together with water soluble thread. Advantageously the sections are joined together by non-woven soluble material such as paper or polymer fibres which are joined with either each section of the bandage by needling or alternatively adhesive. This provides the advantage of a long bandage being produced with multiple sections which can easily be broken apart by water. In the case of the sections of soluble material this gives a significant break between the sections of bandage.

### BRIEF DESCRIPTION OF THE DRAWINGS

Examples of wound dressings made in accordance with the present invention will now be described hereinbelow with reference to the accompanying drawings, in which:

FIG. 1 shows a top plan view of a plaster;

FIG. 2 shows a cross-sectional side view of the plaster in FIG. 1;

FIG. 3 shows a bottom view of a plaster with a removable film present;

FIG. 4 shows a top plan view of a cannula dressing; and

FIG. 5 shows top views of bandages made in accordance with the present invention.

### DETAILED DESCRIPTION

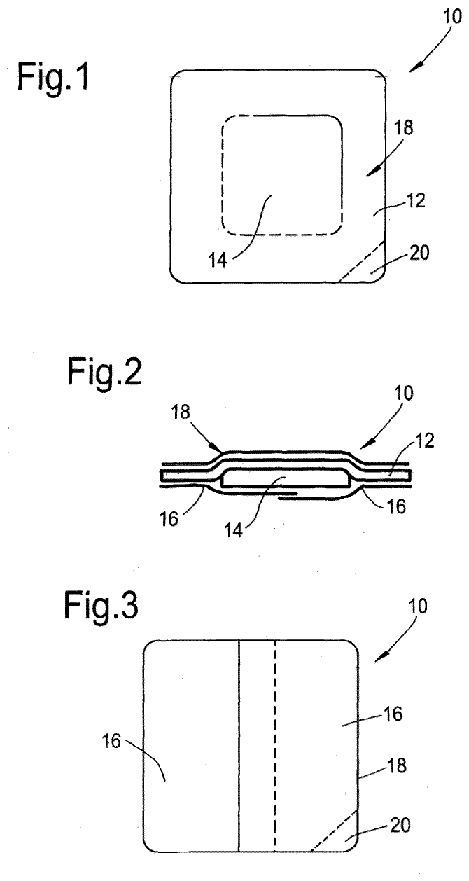

FIG. 1 shows a plaster 10. In this instance the plaster is a roughly square shape however obviously different shapes are equally possible. The plaster 10 comprises on its top side a dissolvable paper or a dissolvable polymer 12. Dissolvable paper made from Sodium Carboxy Methyl Cellulose and wood/wooden pulp, such as that produced by Aquasol® Paper is suitable. Dissolvable polymer made from poly vinyl alcohol, PVA, such as 100% cross laid 38 gsm dissolving PVA is suitable. Preferably the PVA is partially hydrolyzed PVA (e.g. ˜87-89% hydrolyzed) and so dissolves more easily in cold or room-temperature water. Less preferably but alternatively, the PVA is fully hydrolyzed PVA (e.g. ˜ 98-99% hydrolyzed) but this typically requires hot water (typically >85° C.) to dissolve properly. In the centre of the plaster 10 there is seen a raised portion under which is present an absorbent pad 14. The absorbent pad 14 can be a conventional pad or alternatively can be made from absorbent soluble material. The plaster 10 can be covered by a water-resistant film 18 on its intended top surface. The film 18 has a corner 20 with no adhesive.

FIG. 2 shows a cross-section of the plaster 10. The top side the plaster 10 comprises the dissolvable paper or polymer 12, which is covered by the water-resistant film 18 lightly adhered to the dissolvable paper or polymer 12. The underside of the dissolvable paper or polymer 12 has adhesive material on it. The adhesive is selected from a number of readily dissolvable adhesives. The adhesive holds the absorbent pad 14 to the dissolvable paper or polymer 12. Both the dissolvable paper or polymer 12 and the absorbent pad 14 are covered by a foil 16 which covers the bottom of the plaster 10 which faces the patient's skin.

FIG. 3 shows the bottom of the plaster 10 in which it can be seen that the foil 16 has two parts which cover the bottom of the plaster 10 with one half over lapping the other to enable removal of the film 16.

For supply to the public the plaster 10 may be placed in a packet which is sterile. The paper can be selected from ones that are hypoallergenic in order to ensure minimal irritation to a patient's skin.

When the plaster 10 is in use the film 16 is removed from the plaster 10 and the plaster 10 is applied to the cut or wound or skin. When it is desired to remove the plaster 10, the film 18, if present, is peeled back. The plaster 10 is either treated with running water or alternatively has a wet cloth or sponge applied to remove the dissolvable paper or polymer and with care. This will not remove the absorbent pad 14. However, if the absorbent pad is also made of dissolvable material this can be removed at the same time. The absorbent pad 14 can then be removed with as little disturbance of the wound or skin as possible.

Although not shown, the invention may present as a tape made of dissolvable material such as dissolvable paper made from Sodium Carboxy Methyl Cellulose and wood/wooden pulp, such as that produced by Aquasol® Paper is suitable. Alternatively, dissolvable polymer made from poly vinyl alcohol, PVA, such as 100% cross laid 38 gsm dissolving PVA is also suitable and is particularly preferred for cosmetic applications where the tape is used to tape skin or compress skin for selected periods with a view to removing or ameliorating fine lines or wrinkles. The tape may be any size but, in some embodiments is in the form of a pad for placing about the eye or neck or upon the decolletage or hand. The upper side of the tape can be covered by a water-resistant film, ideally with a corner that is adhesive free, to aid removal. The lower side of the tape has adhesive material on it to facilitate attachment to the skin.

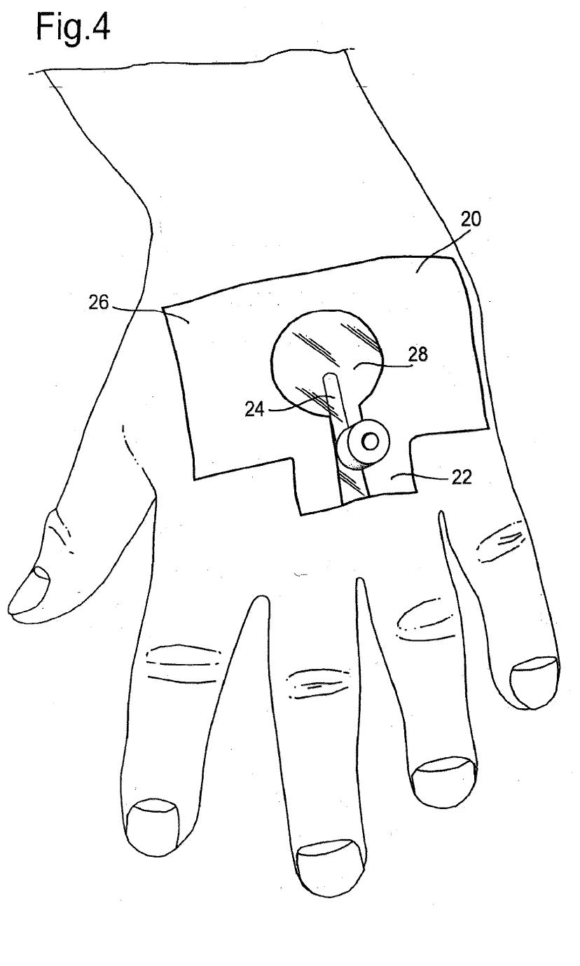

FIG. 4 shows a cannula dressing 20. In this instance, the plaster is a roughly parallelogram shape with an elongated bottom part 22 to cover the cannula 24 but obviously different shapes are equally possible. The cannula dressing 20 comprises on its top side a dissolvable paper or polymer 26. Dissolvable paper is made from Sodium Carboxy Methyl Cellulose and wooden pulp, such as that produce by Aquasol® Paper. Dissolvable polymer is made from poly vinyl alcohol, PVA, such as 100% cross laid 38 gsm dissolving PVA. In the centre of the cannula dressing 20 there is a transparent part 28 which enables the cannula 24 to be seen. In this instance, the transparent part 28 is circular but obviously different shapes are equally possible. The underside of the dissolvable paper or polymer 26 has adhesive material on it. The adhesive is selected from a number of readily dissolvable adhesives. The cannula dressing holds the cannula in place on, in this instance, the left hand. When it is necessary to remove the cannula, a wet sponge, shower or a spray can be used to dissolve the paper, thus allowing the easy removal of the cannula.

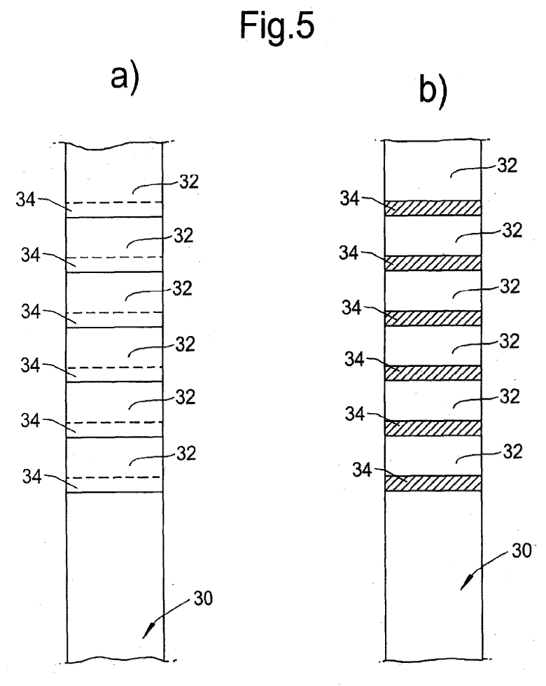

FIG. 5 shows plan views of an elongate bandage 30. Each bandage 30 is comprised of a number of sections 32. The bandage 30 can be of a woven cloth fabric or alternatively of a non-woven fabric. The size of each section 32 of the bandage 30 can be varied and will be dependent upon the intended application. In FIG. 5a the sections 32 are sewn together with soluble thread at a section of soluble material 34. In FIG. 5b there exists a section of soluble material 34 which is joined to each section of bandage 32. The joint between sections here can be formed by needling or alternatively by adhesive or other fixing means. The soluble material may be soluble paper or non-woven soluble paper/wood fibre or polymer thread such as dissolving PVA thread.

Water is applied to the bandage 30 breaking the sections 32 and thus allowing careful removal of each section 32 to occur. Obviously, the bandage of FIG. 5b allows discrete breaks in the bandage 30 and may be suitable for certain purposes where the breakup of the elongate bandage 30 is more required.

What is claimed:

1. A wound dressing, surgical tape or plaster comprising, optionally an absorbent pad, and an adhesive part in which the adhesive part is an adhesive dissolvable paper or polymer.

2. A wound dressing, surgical tape or plaster according to claim 1, in which the adhesive dissolvable paper part covers an adsorbent pad.

3. A wound dressing, surgical tape or plaster according to claim 1, further comprising a removable film or foil covering its adhesive side before use.

4. A wound dressing, surgical tape or plaster according to claim 1, in which the adhesive dissolvable paper or polymer is printed or written or on one side conveying information.

5. A wound dressing surgical tape or plaster according to claim 1, further comprising a removable water resistance film.

6. A wound dressing surgical tape or plaster according to claim 5, in which the removable water resistance film is secured to the topside of the wound dressing surgical tape or plaster via adhesive.

7. A wound dressing surgical tape or plaster according to claim 1, in which the absorbent pad of the wound dressing is made of water-soluble material.

8. A wound dressing surgical tape or plaster according to claim 1, in which the wound dressing surgical tape or plaster is a cannula dressing.

9. A wound dressing surgical tape or plaster according to claim 8, in which the cannula dressing is made from two forms of soluble material.

10. A wound dressing surgical tape or plaster according to claim 8, in which the cannula dressing can be made to any size or shape.

11. A wound dressing surgical tape or plaster according to claim 8, in which the cannula dressing can be secured to the cannula site.

12. A wound dressing, surgical tape or plaster according to claim 1, wherein the dissolvable paper is made from Sodium Carboxy Methyl Cellulose and wood/wooden pulp, or the dissolvable polymer is made from poly vinyl alcohol, PVA.

13. A wound dressing, surgical tape or plaster according to claim 12 wherein said PVA is a cross laid fabric.

14. A wound dressing, surgical tape or plaster according to claim 13 wherein said fabric is about 38 gsm.

15. A wound dressing, surgical tape or plaster according to claim 12 wherein said PVA is partially hydrolyzed PVA (e.g. ˜87-89% hydrolyzed).

16. A wound dressing, surgical tape or plaster according to claim 1 which is sized and shaped to be applied to or about a body part.
